(19) United States
(12) Patent Application Publication
(10) Pub. No.: US 2018/0092631 A1
(43) Pub. Date: Apr. 5, 2018

(54) Blood Vessel Analysis Device and Operating Method Thereof

(71) Applicant: National Kaohsiung University of Applied Sciences, Kaohsiung (TW)

(72) Inventor: Jian-Chiun Liou, Kaohsiung (TW)

(21) Appl. No.: 15/283,468

(22) Filed: Oct. 3, 2016

(51) Int. Cl. A61B 8/08; A61B 5/026; A61B 5/00; A61B 5/1455; A61B 8/00

(52) U.S. Cl. CPC A61B 8/5261; A61B 5/0261; A61B 5/0013; A61B 8/0891; A61B 2562/0238; A61B 5/14552; A61B 8/463; A61B 5/0024; A61B 8/5223

(57) ABSTRACT

A blood vessel analysis device includes a photoelectric detector, a processor and an ultrasonic detector. The photoelectric detector detects a blood vessel and generates a detecting signal. The processor is connected to the photoelectric detector for receiving the detecting signal. The processor converts the detecting signal into a blood flow information specifying a blood flow condition of the blood vessel, and outputs a driving signal when the specified blood flow condition matches a reference blood flow condition. The ultrasonic detector is connected to the processor. The ultrasonic detector detects the blood vessel and generates a blood vessel diameter information upon reception of the driving signal. Based on this, the detection efficiency is improved. An operating method of the blood vessel analysis device is also provided.

### BACKGROUND OF THE INVENTION

### 1. Field of the Invention

The present disclosure generally relates to a blood vessel analysis device and an operating method thereof and, more particularly, to a blood vessel analysis device and an operating method thereof which perform a vascular analysis through photoelectric detection before an ultrasonic detection is performed.

### 2. Description of the Related Art

Vascular abnormalities are a sign of the diseases such as cerebrovascular accident, high blood pressure and heart disease. Each of the above diseases has a high death rate among all the diseases. If the disease can be timely handled when the vascular ailment appears, the risks resulting from vascular abnormalities can be reduced.

In general, when detecting the vascular abnormality of a patient, the medical staff uses an ultrasonic imaging device to perform a vascular analysis on a predetermined part of the blood vessel of the patient. This can determine whether an embolism has occurred on the predetermined part of the blood vessel of the patient. However, there are tens of thousands of blood vessels in human body. Thus, before the measurement of the ultrasonic imaging device, if no any other pretreatment device is used (or no measure is taken) to determine the location where the vascular abnormality has occurred, the medical staff will have to examine the blood vessels one by one with the ultrasonic imaging device. The numerous and repeated examining tasks cause mental and physical exhaustion on both the medical staff and the patient, leading to low detection efficiency.

To overcome the problem of low examining efficiency resulting from the lack of a proper pretreatment device or method, it is necessary to provide a blood vessel analysis device and an operating method of the blood vessel analysis device.

### SUMMARY OF THE INVENTION

It is therefore the objective of this disclosure to provide a blood vessel analysis device and an operating method thereof. With the blood vessel analysis device and its operating method, the location where the vascular abnormality has occurred can be determined through the photoelectric detection, and the ultrasonic detection can be subsequently performed to examine the blood vessel. Thus, the detection efficiency can be improved.

In an embodiment, a blood vessel analysis device includes a photoelectric detector, a processor and an ultrasonic detector. The photoelectric detector is configured to detect a blood vessel and to generate a detecting signal. The processor is electrically connected to the photoelectric detector for receiving the detecting signal. The processor is configured to convert the detecting signal into a blood flow information specifying a blood flow condition of the blood vessel, and to output a driving signal when the specified blood flow condition matches a reference blood flow condition. The ultrasonic detector is electrically connected to the processor. The ultrasonic detector is configured to detect the blood vessel and to generate a blood vessel diameter information upon reception of the driving signal. Based on this, the rough location of the vascular abnormality can be determined through the photoelectric detector, and the processor can drive the ultrasonic detector to examine the blood vessel when the blood vessel is examined to be in an abnormal condition. As such, the detection efficiency is improved.

In a form shown, the blood flow information includes a blood speed value, and the reference blood flow condition includes a reference blood speed value. Based on this, the processor can find out the rough location of the vascular abnormality according to the detected result of the photoelectric detector, and output the driving signal when the blood vessel is examined to be in an abnormal condition. As such, the ultrasonic detector can quickly examine whether the blood vessel has an abnormal condition, improving the detection efficiency.

In another form shown, the blood flow information includes an oxygen content, and the reference blood flow condition includes a reference oxygen content. Based on this, the processor can find out the rough location of the vascular abnormality according to the detected result of the photoelectric detector, and output the driving signal when the blood vessel is examined to be in an abnormal condition. As such, the ultrasonic detector can quickly examine whether the blood vessel has an abnormal condition, improving the detection efficiency.

In the form shown, the blood vessel analysis device further includes a display electrically connected to the processor. The processor receives and outputs the blood flow information or the blood vessel diameter information to the display for display purpose. As such, the related personnel (the medical staff or the patient) are able to view the detected results of the photoelectric detector and the ultrasonic detector through the display, providing a convenient use of the blood vessel analysis device.

In the form shown, the photoelectric detector includes a photoelectric substrate, a light emitting unit and a light detector. The photoelectric substrate includes a surface that is divided into an emission area and a reception area. The light emitting unit is arranged on the emission area and is configured to emit at least one light towards the blood vessel. The light detector is arranged on the emission area and is configured to receive at least one reflected light reflected from the blood vessel. The light detector generates the detecting signal based on the at least one reflected light. As such, the photoelectric detector can generate the detecting signal through the detection of light, attaining smooth generation of the detecting signal.

In the form shown, the light emitting unit includes a plurality of light emitting sections capable of emitting a plurality of lights with different wavelengths. Each of the plurality of light emitting sections includes at least one micro light-emitting diode. As such, the light emitting unit can emit different kinds of wavelengths of lights. When the light emitting unit emits the lights towards the blood vessel of the patient, although some of the lights may not be able to penetrate the body tissue due to its wavelength, the other light(s) is still able to reach the blood vessel. Thus, the detecting signal can be smoothly generated.

In the form shown, the blood vessel analysis device further includes a controller electrically connected to the light emitting unit. The controller is configured to control the plurality of light emitting sections to emit the plurality of lights in sequence along a direction. As such, when the light emitting sections emit different wavelengths of lights towards the blood vessel in sequence, the light detector can receive the reflected lights (reflected from the blood vessel) one after another. Accordingly, the light detector can generate the detecting signal based on the reflected lights. For example, the light detector can generate the detecting signal by receiving only a certain wavelength(s) of the light(s) or based on the order the reflected lights are received. As such, the accuracy of measurement is improved.

Alternatively, the controller is configured to control the plurality of light emitting sections to emit the plurality of lights in a random manner. As such, when the light emitting sections emit different wavelengths of lights towards the blood vessel in sequence, the light detector can receive the reflected lights (reflected from the blood vessel) in a random manner. Accordingly, the light detector can generate the detecting signal based on the reflected lights. For example, the light detector can generate the detecting signal by receiving only a certain wavelength(s) of the light(s) or based on the randomness of the reflected lights. As such, the accuracy of measurement is improved.

In the form shown, the plurality of light emitting sections includes a red light emitting section, a green light emitting section and a blue light emitting section. As such, the light emitting unit can emit different kinds of wavelengths of lights. When the light emitting unit emits the lights towards the blood vessel of the patient, although some of the lights may not be able to penetrate the body tissue due to its wavelength, the other light(s) is still able to reach the blood vessel. Thus, the detecting signal can be smoothly generated.

In the form shown, each of the at least one micro light-emitting diode has a size of 20 μm by 20 μm. Thus, arrangement of the micro LEDs not only reduces the volume and power consumption of the light emitting unit, but also permits the lights to smoothly reach the blood vessel due to its finer scale. Thus, the detection accuracy is improved.

In the form shown, the photoelectric detector includes a wireless transmission module, and the processor includes a wireless transceiving module electrically connected to the wireless transmission module. Based on this, the processor can be electrically connected to the photoelectric detector via the wireless transceiving module, improving the convenience in data transmission.

In the form shown, each of the wireless transmission module and the wireless transceiving module is a WIFI structure, a zigbee structure or a Bluetooth structure. Based on this, the processor can be electrically connected to the photoelectric detector via the wireless transceiving module, improving the convenience in data transmission.

In another embodiment, an operating method of a blood vessel analysis device includes detecting a blood vessel with a photoelectric detector, generating a detecting signal, receiving the detecting signal with a processor, and executing a determination step to convert the detecting signal into a blood flow information specifying a blood flow condition of the blood vessel, to determine whether the specified blood flow condition matches a reference blood flow condition, to generate a driving signal if the determined result is positive, and to continue the determination step if the determined result is negative. The operating method of the blood vessel analysis device further includes receiving the driving signal with an ultrasonic detector, and detecting the blood vessel with the ultrasonic detector to generate a blood vessel diameter information. Based on this, the rough location of the vascular abnormality can be determined through the photoelectric detector, and the processor can drive the ultrasonic detector to examine the blood vessel when the blood vessel is examined to be in an abnormal condition. As such, the detection efficiency is improved.

In a form shown, the operating method of the blood vessel analysis device further includes providing a display, receiving the blood vessel diameter information with the display, outputting the blood flow information or the blood vessel diameter information from the processor to the display, and displaying the blood flow information or the blood vessel diameter information with the display. Based on this, the display can display the blood flow information or the blood vessel diameter information, permitting the related personnel (the medical staff or the patient) to view the detected results of the photoelectric detector and the ultrasonic detector through the display. As such, convenient use of the blood vessel analysis device can be provided.

### BRIEF DESCRIPTION OF THE DRAWINGS

The present disclosure will become more fully understood from the detailed description given hereinafter and the accompanying drawings which are given by way of illustration only, and thus are not limitative of the present disclosure, and wherein:

FIG. 1 shows a block diagram of a blood vessel analysis device according to an embodiment of the disclosure.

FIG. 2 shows a photoelectric detector of the blood vessel analysis device of the embodiment of the disclosure.

In the various figures of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the disclosure.

### DETAILED DESCRIPTION OF THE INVENTION

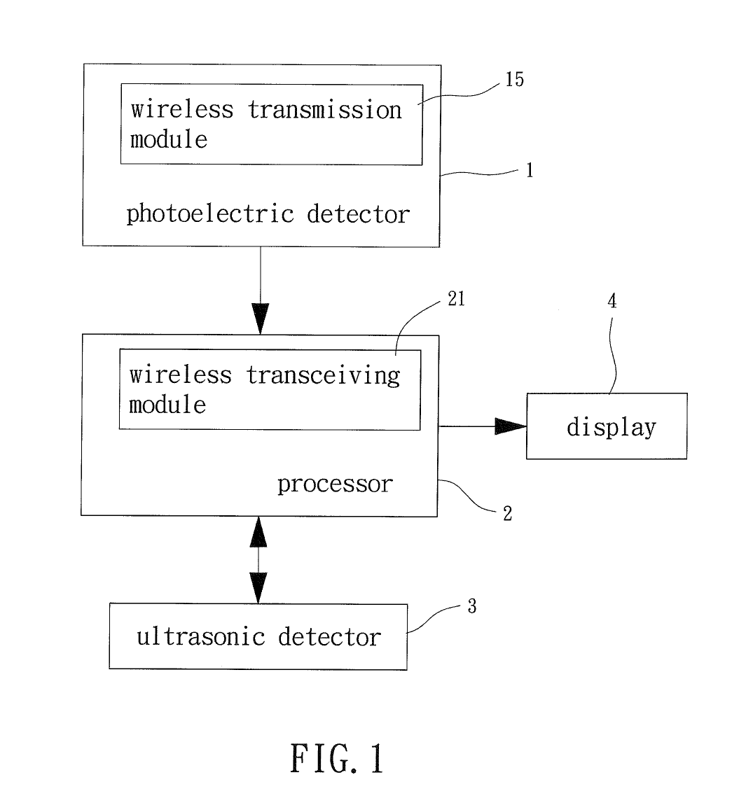

FIG. 1 shows a block diagram of a blood vessel analysis device according to an embodiment of the disclosure. The blood vessel analysis device includes a photoelectric detector 1, a processor 2 and an ultrasonic detector 3. The photoelectric detector 1 and the ultrasonic detector 3 are electrically connected to the processor 2.

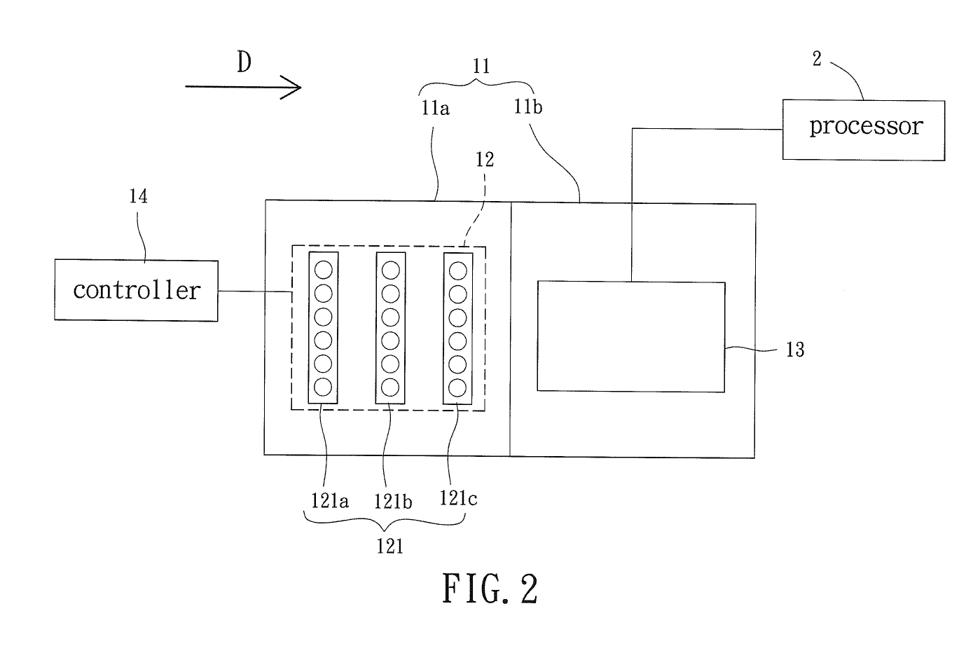

Referring to both FIGS. 1 and 2, the photoelectric detector 1 is used to detect a blood vessel and to generate a detecting signal. In the embodiment, the photoelectric detector 1 includes a photoelectric substrate 11, a light emitting unit 12 and a light detector 13. The photoelectric substrate 1 includes a surface that is divided into an emission area 11a and a reception area 11b. The light emitting unit 12 is arranged on the emission area 11a and emits at least one light towards the blood vessel. The light detector 13 is arranged on the reception area 11b and is used to receive at least one reflected light reflected from the blood vessel. The light detector 13 generates the detecting signal according to the at least one reflected light. In this arrangement, the photoelectric detector 1 can generate the detecting signal through light detection, attaining smooth generation of the detecting signal.

The light emitting unit 12 includes a plurality of light emitting sections 121 arranged on the emission area 11a in intervals along a direction D. Each of the light emitting sections 121 emits a different wavelength of light. The quantity of the plurality of light emitting sections 121 is not limited. In the embodiment, there are three light emitting sections 121, including a red light emitting section 121a, a green light emitting section 121b and a blue light emitting section 121c. As such, the light emitting unit 12 can emit different kinds of wavelengths of lights. When the light emitting unit 12 emits the lights towards the blood vessel of the patient, although some of the lights may not be able to penetrate the body tissue due to its wavelength, the other light(s) is still able to reach the blood vessel. Thus, the detecting signal can be smoothly generated.

Each of the plurality of light emitting sections 121 contains at least one micro light-emitting diode (μLED). The size of the micro LED is 20 μm by 20 μm. Therefore, arrangement of the micro LEDs not only reduces the volume and power consumption of the light emitting unit 12, but also permits the lights to smoothly reach the blood vessel due to its finer scale. Thus, the detection accuracy is improved.

Furthermore, the photoelectric detector 1 may further include a controller 14 electrically connected to the light emitting unit 12. The controller 14 is used to control the light emitting sections 121 to irradiate the lights in sequence in the direction D. In the embodiment, since the light emitting sections 121 include the red light emitting section 121a, the green light emitting section 121b and the blue light emitting section 121c, the controller 14 can control the red light emitting section 121a, the green light emitting section 121b and the blue light emitting section 121c to emit lights in sequence. Thus, when the light emitting sections 121 emit different wavelengths of lights towards the blood vessel in sequence, the light detector 13 can receive the reflected lights (reflected from the blood vessel) one after another. Accordingly, the light detector 13 can generate the detecting signal based on the reflected lights. For example, the light detector 13 can generate the detecting signal by receiving only a certain wavelength(s) of the light(s) or based on the order the reflected lights are received. As such, the accuracy of measurement is improved.

Based on the arrangement of the controller 14 of the photoelectric detector 1, the controller 14 is electrically connected to the light emitting unit 12 and controls the light emitting sections 121 to emit a different wavelength of light in a random manner. In the embodiment, the light emitting sections 121 include the red light emitting section 121a, the green light emitting section 121b and the blue light emitting section 121c. In this regard, the controller 14 can control the red light emitting section 121a, the green light emitting section 121b and the blue light emitting section 121c to emit a red light, a green light or a blue light in a random manner. Based on this, when the light emitting sections 121 emit different wavelengths of lights to the blood vessel in a random manner, the light detector 13 can receive the reflected lights reflected from the blood vessel. As such, the light detector 13 can generate the detecting signal based on the reflected lights. For example, the light detector 13 can generate the detecting signal based a certain wavelength(s) of light(s), or based on the randomness of the reflected lights. As such, the detection accuracy can be improved.

The photoelectric detector 1 preferably includes a wireless transmission module 15, which may be a WIFI structure, a zigbee structure or a Bluetooth structure. As such, the photoelectric detector 1 can be electrically connected to the processor 2 through the wireless transmission module 15, improving the convenience in data transmission.

The processor 2 is electrically connected to the photoelectric detector 1 for receiving the detecting signal. The processor 2 converts the detecting signal into a blood flow information specifying the blood flow condition of the blood vessel, and outputs a driving signal when the specified blood flow condition matches a reference blood flow condition. The processor 2 can be any device with logic calculation and statistical functions. Based on this, the processor 2 can convert the detecting signal into the blood flow information. For example, the processor 2 can determine the ability of the blood to absorb the light, so as to calculate the flow speed or oxygen content of the blood. Then, the blood flow information is compared with the reference flow range.

The blood flow information may contain one or more data values. The reference blood flow condition is preferably set as an abnormal condition. In this embodiment, the blood flow information may contain a blood speed value, and the reference blood flow condition may contain a reference blood speed value. Alternatively, the blood flow information may contain an oxygen content, and the reference blood flow condition may contain a reference oxygen content. Based on the detected result of the photoelectric detector 1, the processor 2 can find out the rough location of the vascular abnormality, and output the driving signal when the blood vessel is examined to be in an abnormal condition. This permits the ultrasonic detector 3 to quickly detect whether the blood vessel has an abnormal condition, improving the detection efficiency.

Moreover, the processor 2 preferably includes a wireless transceiving module 21 electrically connected to the wireless transmission module 15 of the photoelectric detector 1. The wireless transceiving module 21 can be further connected to the ultrasonic detector 3. The wireless transceiving module 21 can be the WIFI structure, the zigbee structure or the Bluetooth structure. Based on this, the processor 2 can be electrically connected to the photoelectric detector 1 via the wireless transceiving module 21, improving the convenience in data transmission.

The ultrasonic detector 3 is electrically connected to the processor 2. After the ultrasonic detector 3 receives the driving signal, the ultrasonic detector 3 detects the blood vessel and generates a blood vessel diameter information. The ultrasonic detector 3 can be any device capable of performing angiography, and can calculate the diameter of the blood vessel during the angiography. The calculated diameter of the blood vessel can be used as the blood vessel diameter information. The ultrasonic detector 3 even can output different warning messages when the diameter of the blood vessel is respectively larger and smaller than a reference diameter. As such, the blood vessel analysis device of the disclosure can find out the rough location of the vascular abnormality through the photoelectric detector 1, and the processor 2 can drive the ultrasonic detector 3 to accurately examine the condition of the blood vessel. Advantageously, the detection accuracy is improved.

The blood vessel analysis device of the disclosure can further include a display 4 electrically connected to the processor 2. The processor 2 can receive the blood vessel diameter information and output the blood vessel diameter information to the display 4. The display 4 can display the blood vessel diameter information. Thus, the display 4 can be used to display the blood flow information or the blood vessel diameter information, so that the related personnel (the medical staff or the patient) are able to view the detected results of the photoelectric detector 1 and the ultrasonic detector 3 through the display 4. Convenient use of the blood vessel analysis device is attained.

Based on the aforementioned structure of the blood vessel analysis device, an operating method of the blood vessel analysis device includes the steps as stated below.

In a first step, a photoelectric detector 1 is used to detect the blood vessel, thus generating the detecting signal. As such, the photoelectric detector 1 can generate the detecting signal through a light detector, attaining smooth generation of the detecting signal.

In a second step, the processor 2 is used to receive the detecting signal and to execute a determination step. In the determination step, the processor 2 converts the detecting signal into the blood flow information specifying the blood flow condition of the blood vessel, and determines whether the specified blood flow condition matches the reference blood flow condition. If the determined result is positive, the driving signal is generated and outputted. If the determined result is negative, the determination step is continued. The detail of the processor 2 executing the determination step is described above, and therefore is not repeated herein. Based on this, the processor 2 can find out the rough location of the vascular abnormality according to the detected result of the photoelectric detector 1, and output the driving signal when the blood vessel is examined to be in an abnormal condition. As such, the ultrasonic detector 3 can quickly examine whether the blood vessel has an abnormal condition, improving the detection efficiency.

In a third step, the ultrasonic detector 3 is used to receive the driving signal, so that the ultrasonic detector 3 can detect the blood vessel and generate the blood vessel diameter information. Based on this, the rough location of the vascular abnormality can be determined through the photoelectric detector 1, and the processor 2 can drive the ultrasonic detector 3 to examine the blood vessel when the blood vessel is examined to be in an abnormal condition. As such, the detection efficiency is improved.

In a fourth step, a display 4 is provided, and the blood vessel diameter information is received by the processor 2. In this regard, the processor 2 outputs the blood flow information or the blood vessel diameter information to the display 4 for display purpose. As such, the related personnel (the medical staff or the patient) are able to view the detected results of the photoelectric detector 1 and the ultrasonic detector 3 through the display 4. Convenient use of the blood vessel analysis device is attained.

In conclusion, the blood vessel analysis device and its operating method can find out the rough location where the vascular abnormality has occurred, and drive the ultrasonic detector 3 to examine the blood vessel through the processor 2. Thus, the detection efficiency is improved.

Although the disclosure has been described in detail with reference to its presently preferable embodiments, it will be understood by one of ordinary skill in the art that various modifications can be made without departing from the spirit and the scope of the disclosure, as set forth in the appended claims.

What is claimed is:

1. A blood vessel analysis device comprising:
a photoelectric detector configured to detect a blood vessel and to generate a detecting signal;
a processor electrically connected to the photoelectric detector for receiving the detecting signal, wherein the processor is configured to convert the detecting signal into a blood flow information specifying a blood flow condition of the blood vessel, and to output a driving signal when the specified blood flow condition matches a reference blood flow condition; and
an ultrasonic detector electrically connected to the processor, wherein the ultrasonic detector is configured to detect the blood vessel and to generate a blood vessel diameter information upon reception of the driving signal.

2. The blood vessel analysis device as claimed in claim 1, wherein the blood flow information comprises a blood speed value, and the reference blood flow condition comprises a reference blood speed value.

3. The blood vessel analysis device as claimed in claim 1, wherein the blood flow information comprises an oxygen content, and the reference blood flow condition comprises a reference oxygen content.

4. The blood vessel analysis device as claimed in claim 2, wherein the blood flow information comprises an oxygen content, and the reference blood flow condition comprises a reference oxygen content.

5. The blood vessel analysis device as claimed in claim 1, further comprising a display electrically connected to the processor, wherein the processor receives and outputs the blood flow information or the blood vessel diameter information to the display for display purpose.

6. The blood vessel analysis device as claimed in claim 1, wherein the photoelectric detector comprises a photoelectric substrate, a light emitting unit and a light detector, wherein the photoelectric substrate comprises a surface that is divided into an emission area and a reception area, wherein the light emitting unit is arranged on the emission area and is configured to emit at least one light towards the blood vessel, wherein the light detector is arranged on the emission area and is configured to receive at least one reflected light reflected from the blood vessel, and wherein the light detector generates the detecting signal based on the at least one reflected light.

7. The blood vessel analysis device as claimed in claim 6, wherein the light emitting unit comprises a plurality of light emitting sections capable of emitting a plurality of lights with different wavelengths, wherein each of the plurality of light emitting sections comprises at least one micro light-emitting diode.

8. The blood vessel analysis device as claimed in claim 7, further comprising a controller electrically connected to the light emitting unit, wherein the controller is configured to control the plurality of light emitting sections to emit the plurality of lights in sequence along a direction.

9. The blood vessel analysis device as claimed in claim 7, further comprising a controller electrically connected to the light emitting unit, wherein the controller is configured to control the plurality of light emitting sections to emit the plurality of lights in a random manner.

10. The blood vessel analysis device as claimed in claim 7, wherein the plurality of light emitting sections comprises a red light emitting section, a green light emitting section and a blue light emitting section.

11. The blood vessel analysis device as claimed in claim 7, wherein each of the at least one micro light-emitting diode has a size of 20 μm by 20 μm.

12. The blood vessel analysis device as claimed in claim 1, wherein the photoelectric detector comprises a wireless transmission module, wherein the processor comprises a wireless transceiving module electrically connected to the wireless transmission module.

13. The blood vessel analysis device as claimed in claim 12, wherein each of the wireless transmission module and the wireless transceiving module is a WIFI structure, a zigbee structure or a Bluetooth structure.

14. An operating method of a blood vessel analysis device, comprising:
detecting a blood vessel with a photoelectric detector;
generating a detecting signal;
receiving the detecting signal with a processor;
executing a determination step to convert the detecting signal into a blood flow information specifying a blood flow condition of the blood vessel, to determine whether the specified blood flow condition matches a reference blood flow condition, to generate a driving signal if the determined result is positive, and to continue the determination step if the determined result is negative;
receiving the driving signal with an ultrasonic detector; and
detecting the blood vessel with the ultrasonic detector to generate a blood vessel diameter information.

15. The operating method of the blood vessel analysis device as claimed in claim 14, further comprising:
providing a display;
receiving the blood vessel diameter information with the display;
outputting the blood flow information or the blood vessel diameter information from the processor to the display; and
displaying the blood flow information or the blood vessel diameter information with the display.
